(19) United States
(12) Patent Application Publication
(10) Pub. No.: US 2021/0229990 A1
(43) Pub. Date: Jul. 29, 2021

(54) FLUIDIZED COKING WITH CARBON CAPTURE AND CHEMICAL PRODUCTION

(71) Applicant: ExxonMobil Research and Engineering Company, Annandale, NJ (US)

(72) Inventor: Mohsen N. Harandi, New Hope, PA (US)

(21) Appl. No.: 16/774,080

(22) Filed: Jan. 28, 2020

(51) Int. Cl. C01B 3/30; C01B 32/40

(52) U.S. Cl. CPC C01B 3/30; C01B 32/40; C01B 2203/0283; C01B 2203/0233; C01B 2203/0238; C01B 2203/048; C01B 2203/065; C01B 2203/068; C01B 2203/0475; C01B 2203/0485; C01B 2203/061

(57) ABSTRACT

Systems and methods are provided for improving the integration of fluidized coking systems that include an associated gasifier with other refinery and/or chemical plant processes. The improved integration can be based on one or more types of integration improvements. In some aspects, the integration can allow for improved carbon capture. In other aspects, the integration can allow for production of higher quality synthesis gas, which can then facilitate production of various chemicals, such as ammonia or urea. In still other aspects, the integration can allow for incorporation of H₂S generated during the fluidized coking and gasification into a fertilizer product. In yet other aspects, the integration can allow the fluidized coking system to continue to operate even when the associated refinery and/or chemicals production processes are off-line. In still other aspects, the integration can allow two or more of the above integration advantages, or three or more, such as up to all of the above integration advantages.

### FIELD

Systems and methods are provided for mitigating CO₂ production from fluidized coking as well as producing chemicals from the resulting fuel gas generated by gasification of the fluidized coke.

### BACKGROUND

Coking is a carbon rejection process that is commonly used for upgrading of heavy oil feeds and/or feeds that are challenging to process, such as feeds with a low ratio of hydrogen to carbon. In addition to producing a variety of liquid products, typical coking processes can also generate a substantial amount coke. Because the coke contains carbon, the coke is potentially a source of additional valuable products in a refinery setting. However, fully realizing this potential remains an ongoing challenge.

Coking processes in modern refinery settings can typically be categorized as delayed coking or fluidized bed coking. Fluidized bed coking is a petroleum refining process in which heavy petroleum feeds, typically the non-distillable residues (resids) from the fractionation of heavy oils are converted to lighter, more useful products by thermal decomposition (coking) at elevated reaction temperatures, typically 480° C. to 590° C., (900° F. to 1100° F.) and in most cases from 500° C. to 550° C. (930° F. to 1020° F.). Heavy oils which may be processed by the fluid coking process include heavy atmospheric resids, petroleum vacuum distillation bottoms, aromatic extracts, asphalts, and bitumens from tar sands, tar pits and pitch lakes of Canada (Athabasca, Alta.), Trinidad, Southern California (La Brea (Los Angeles), McKittrick (Bakersfield, Calif.), Carpinteria (Santa Barbara County, Calif.), Lake Bermudez (Venezuela) and similar deposits such as those found in Texas, Peru, Iran, Russia and Poland.

The Flexicoking™ process, developed by Exxon Research and Engineering Company, is a variant of the fluid coking process that is operated in a unit including a reactor and a heater, and including a gasifier for gasifying the coke product by reaction with an air/steam mixture to form a low heating value fuel gas. A stream of coke passes from the heater to the gasifier where all but a small fraction of the coke is gasified to a gas containing a relatively low BTU content, such as ^{˜}120 BTU/standard cubic feet, by the addition of steam and air in a fluidized bed in an oxygen-deficient environment to form fuel gas comprising carbon monoxide and hydrogen. In a typical Flexicoking™ configuration, the fuel gas product from the gasifier, containing entrained coke particles, is returned to the heater to provide most of the heat required for thermal cracking in the reactor with the balance of the reactor heat requirement supplied by combustion in the heater. A small amount of net coke (e.g., ˜1 percent of feed) is withdrawn from the heater to purge the system of metals and ash. The fuel gas product is withdrawn from the heater following separation in internal cyclones which return coke particles through their diplegs.

Examples of Flexicoking process are described in patents of Exxon Research and Engineering Company, including, for example, U.S. Pat. No. 3,661,543 (Saxton), U.S. Pat. No. 3,759,676 (Lahn), U.S. Pat. No. 3,816,084 (Moser), U.S. Pat. No. 3,702,516 (Luckenbach), U.S. Pat. No. 4,269,696 (Metrailer). A variant is described in U.S. Pat. No. 4,213,848 (Saxton) in which the heat requirement of the reactor coking zone is satisfied by introducing a stream of light hydrocarbons from the product fractionator into the reactor instead of the stream of hot coke particles from the heater. Another variant is described in U.S. Pat. No. 5,472,596 (Kerby) using a stream of light paraffins injected into the hot coke return line to generate olefins. Early work proposed units with a stacked configuration but later units have migrated to a side-by-side arrangement.

Although the fuel gas from the gasifier can be used for heating, due to the low energy content, burning of the fuel gas for heat can still represent a relatively low value use for the carbon in the fuel gas. Additionally, due to the relatively high CO and CO₂ content in the fuel gas, the resulting combustion exhaust from burning of the fuel gas can represent a substantial portion of the CO₂ emissions for a refinery complex. What is needed are systems and methods that can allow for generation of still higher economic value products from the gasifier associated with a Flexicoking™ process, while also reducing or minimizing exhaust of CO₂ to the atmosphere.

U.S. Pat. No. 9,234,146 describes a process for gasification of heavy residual oil and coke from a delayed coker unit. The gasification allows for production of synthesis gas from the heavy residual oil and coke. The gasifier used in the process corresponds to a membrane wall gasifier that uses an internal cooling screen that is protected by a layer of refractory material. The combination of the cooling screen and the layer of refractory material allows the slag formed during gasification to solidify and flow downward to the quench zone at the bottom of the reactor.

U.S. Pat. No. 7,919,065 describes systems and methods for producing ammonia and Fischer-Tropsch liquids based on gasification of a slurry of coal solids or petroleum coke. Slag is produced in the gasifier as a side product during gasification.

U.S. Pat. No. 10,400,177 describes methods for upgrading the fuel gas generated by a gasifier associated with a fluidized coking system. The upgraded products can include oligomerized products and/or methanol.

U.S. Pat. No. 10,407,631 describes methods for producing methanol, ammonia, and/or urea by upgrading the fuel gas generated by a gasifier associated with a fluidized coking system. In some aspects, the gasification can be performed using an enriched oxygen-containing stream, such as an oxygen-containing stream formed by an air separation unit.

### SUMMARY

In various aspects, a method for producing synthesis gas or products derived from synthesis gas is provided. The method includes exposing a feedstock comprising a T10 distillation point of 343° C. or more to a fluidized bed comprising solid particles in a reactor under thermal cracking conditions to form at least a 343° C.− liquid product, the thermal cracking conditions comprising 10 wt % or more conversion of the feedstock relative to 343° C., the thermal cracking conditions being effective for depositing coke on the solid particles. The method further includes introducing an oxygen-containing stream, a hydrocarbon-containing stream, and steam into a gasifier. The method further includes passing at least a portion of the solid particles comprising deposited coke from the reactor to the gasifier, the solid particles optionally comprising coke. The method further includes exposing the at least a portion of the solid particles comprising deposited coke to gasification conditions in the gasifier to form a gas phase product comprising H₂, CO, and CO₂ and partially gasified coke particles, the gasification conditions further comprising conditions for at least one of reforming and gasifying hydrocarbons in the hydrocarbon-containing stream in the presence of the steam, the gas phase product comprising a combined volume of H₂ and CO that is greater than 70% (or greater than 140%) of a volume of N₂ in the gas phase product. The method further includes removing at least a first portion of the partially gasified coke particles from the gasifier. Additionally, the method includes passing at least a second portion of the partially gasified coke particles from the gasifier to the reactor.

In various aspects, a method for producing synthesis gas or products derived from synthesis gas is provided. The method includes exposing a feedstock comprising a T10 distillation point of 343° C. or more to a fluidized bed comprising solid particles in a reactor under thermal cracking conditions to form a 343° C.− liquid product, the thermal cracking conditions comprising 10 wt % or more conversion of the feedstock relative to 343° C., the thermal cracking conditions being effective for depositing coke on the solid particles, the solid particles optionally comprising coke. The method further includes introducing steam and a stream comprising O₂ and N₂ into a gasifier. The method further includes passing at least a portion of the solid particles comprising deposited coke from the reactor to the gasifier. The method further includes exposing the at least a portion of the solid particles comprising deposited coke to gasification conditions in the gasifier to form a gas phase product comprising H₂, N₂, CO, and CO₂ and partially gasified coke particles. The method further includes removing at least a first portion of the partially gasified coke particles from the gasifier. The method further includes passing at least a second portion of the partially gasified coke particles from the gasifier to the reactor. The method further includes separating, during a first time period, CO₂ from at least a portion of the gas phase product to form a dilute synthesis gas stream. The method further includes separating, during the first time period, N₂ from the dilute synthesis gas stream to form a nitrogen-containing stream and a synthesis gas stream. The method further includes exposing, during the first time period, at least a portion of the synthesis gas stream to a catalyst in a synthesis reactor to form a chemical product, the chemical product optionally comprising at least one of methanol and ammonia. The method further includes combining, during the first time period, a hydrocarbon-containing stream with the nitrogen-containing stream to form a low-BTU gas, at least a portion of the low-BTU gas being passed as a fuel or reagent to an additional process, the hydrocarbon-containing stream optionally further comprising H₂. The method further includes stopping, during a second time period, the operation of the synthesis reactor. Additionally, the method includes passing, during the second time period, at least a portion of the gas phase product to the additional process.

### BRIEF DESCRIPTION OF THE DRAWINGS

FIG. 1 shows an example of a fluidized bed coking system including a coker, a heater, and a gasifier.

FIG. 2 shows an example of a fluidized bed coking system including a coker and a gasifier.

FIG. 3 shows an example of a configuration for integrating fluidized coking with production of methanol, ammonia, and/or other products derived at least in part from a synthesis gas.

FIG. 4 shows an example of a configuration for integrating fluidized coking with systems for carbon capture, sulfur removal, and production of chemicals.

FIG. 5 shows still another example of a configuration for integrating fluidized coking with systems for carbon capture, sulfur removal, and production of chemicals.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, systems and methods are provided for improving the integration of fluidized coking systems that include an associated gasifier with other refinery and/or chemical plant processes. The improved integration can be based on one or more types of integration improvements. In some aspects, the integration can allow for improved carbon capture. In other aspects, the integration can allow for production of higher quality synthesis gas, which can then facilitate production of various chemicals, such as ammonia or urea. In still other aspects, the integration can allow for incorporation of H₂S generated during the fluidized coking and gasification into a fertilizer product. In yet other aspects, the integration can allow the fluidized coking system to continue to operate even when the associated refinery and/or chemicals production processes are off-line. In still other aspects, the integration can allow two or more of the above integration advantages, or three or more, such as up to all of the above integration advantages.

Some integration advantages can be related to producing high quality synthesis gas from a fluidized coking system that includes an integrated gasifier. One option for improving the quality of the synthesis gas can be to reduce the nitrogen content in the gasifier, such as by using an oxygen-containing gas that has a lower nitrogen content. Another option for improving the quality of the synthesis gas can be to add additional types of feed components to the gasifier environment, so that steam reforming and/or gasification of hydrocarbons richer in H₂ than coke can also occur within the gasifier environment. For example, addition of methane and more steam to the gasifier increases H₂ content of the produced fuel gas.

Additionally or alternately, systems and methods are provided for integrating a fluidized coking process, a coke gasification process, and processes for production of compounds from the synthesis gas generated during the coke gasification.

It is noted that integration of a fluidized coking system with chemical production can also provide advantages related to reduced refinery footprint. For example, in configurations involving ammonia production, by converting H₂-rich hydrocarbons with steam in the gasifier, the need for a separate reforming unit to produce H₂ can be reduced, minimized, or eliminated. The need for a demethanator can also be avoided. Similar types of equipment footprint benefits can be achieved for configurations for production of other chemicals, such as methanol, urea, or fertilizer.

In this discussion, some feeds, fractions, or products may be described based on a fraction that boils below or above a specified distillation point. For example, a 343° C.− product corresponds to a product that substantially contains components with a boiling point (at standard temperature and pressure) of 343° C. or less. Similarly, a 343° C.+ product corresponds to a product that substantially contains components with a boiling point of 343° C. or more. Substantially containing components within a boiling range is defined herein as containing 90 vol % or more of components within the boiling range, optionally 95 vol % or more, such as a product where all components are within the specified boiling range.

In this discussion, a liquid product is defined as a product that is substantially in the liquid phase at 20° C. and ˜100 kPa-a. Similarly, a gas product is defined as a product that is substantially in the gas phase at 20° C. and ˜100 kPa-a.

In this discussion, reference may be made to conversion of a feedstock relative to a conversion temperature. Conversion relative to a temperature can be defined based on the portion of the feedstock that boils at greater than the conversion temperature. The amount of conversion during a process (or optionally across multiple processes) can correspond to the weight percentage of the feedstock converted from boiling above the conversion temperature to boiling below the conversion temperature. As an illustrative hypothetical example, consider a feedstock that includes 40 wt % of components that boil at 650° F. (˜343° C.) or greater. By definition, the remaining 60 wt % of the feedstock boils at less than 650° F. (˜343° C.). For such a feedstock, the amount of conversion relative to a conversion temperature of ˜343° C. would be based only on the 40 wt % that initially boils at ˜343° C. or greater. If such a feedstock could be exposed to a process with 30% conversion relative to a ˜343° C. conversion temperature, the resulting product would include 72 wt % of ˜343° C.− components and 28 wt % of ˜343° C.+ components.

In this discussion, a low BTU gas is defined as a gas having an energy content of 360 BTU/standard cubic foot or less (˜10.5 kJ/m³ or less).

### Increased Synthesis Gas Quality

One difficulty with upgrading fuel gas from a gasifier to higher value products is the relatively low content of synthesis gas in the fuel gas. In some aspects, the quality of the fuel gas can be increased by using the gasifier environment to perform additional H₂ generation reactions. By producing more H₂ in the gasifier environment, in combination with using a water gas shift catalyst to convert a portion of the H₂O and CO the in the environment to CO₂ and H₂, a fuel gas can be generated with a substantially increased synthesis gas content while also increasing CO₂ concentration of the gas. Increasing the CO₂ concentration can improve the economics for performing carbon capture on the gas versus simply burning the stream with air at a lower pressure in refinery furnaces.

A gasification zone for a gasifier associated with a fluidized coker is typically maintained at a high temperature ranging from 850° C. to 1000° C. (1560° F. to 1830° F.) and a pressure ranging from 0 kPag to 1000 kPag (0 psig to 150 psig), preferably from 200 kPag to 400 kPag (30 psig to 60 psig). Conventionally, in addition to coke particles, steam and an oxygen-containing gas are passed into the gasification environment. Under conventional conditions, this can allow for combustion of a sufficient amount of coke from the coke particles to provide the heat for the gasifier environment as well as at least a portion of the heat for the associated fluid coking environment.

Inputs to the gasification environment can be modified when forming a fuel gas with an increased synthesis gas content. One modification can be to introduce a hydrocarbon into the gasification environment. Methane is an example of a suitable hydrocarbon, but any convenient hydrocarbon or mixture of hydrocarbons that is suitable for gasification and/or reforming could be used. Natural gas is another example of a hydrocarbon input that could be introduced into the gasification environment. Optionally, the hydrocarbon input can be distributed in a relatively even manner in at least one radial one, such as a middle radial zone of the gasifier. In such aspects, the oxygen for the gasifier can be introduced in a different zone, such as a lower radial zone. This can facilitate combustion of coke in preference to combustion of hydrocarbon, thus ensuring sufficient coke burning to minimize coke rejection and balance the coking process. In some aspects, the hydrocarbon and steam addition rates can be adjusted to maintain the desired ratio of H₂ to N₂ in the fuel gas for the ammonia plant. In some aspects, a preferred ratio of H₂ to N₂ is roughly 1.5, to eliminate need to separate N₂ from the gasification air or the fuel gas prior to using the fuel gas for ammonia production. If it is desired to make methanol or its derivatives then the appropriate stoichiometry ratio of H₂ to CO can be used to optimize the gasification operations.

In addition to adding a hydrocarbon input to the gasification environment, the amount of oxygen in the environment can also be reduced to substantially below the stoichiometric amount that would be needed for complete combustion of the coke particles and the hydrocarbon input. The amount of steam can also be substantially increased. This combination of modifications to the input flows to the gasifier can further contribute to control of H₂ and CO in the gasifier. By providing a substoichiometric amount of oxygen, insufficient oxidant is available to combust the available fuel. In some aspects, the flow rate of O₂ introduced into the gasifier can correspond to 45% to 75% of the O₂ that would be required for complete combustion of all coke plus hydrocarbon, based on the respective flow rates of coke and hydrocarbon into the gasifier. Introducing extra steam can facilitate a water gas shift reaction, so that a portion of the CO produced by combustion is converted to H₂. This can assist with producing a more desirable ratio of H₂ to CO in the resulting synthesis gas in the gasifier output stream.

In some aspects, the oxygen-containing gas can be an oxygen-containing gas having a low nitrogen content, such as oxygen from an air separation unit or another oxygen stream including 95 vol % or more of oxygen, or 98 vol % or more, are passed into the gasifier for reaction with the solid particles comprising coke deposited on them in the coking zone. A separate diluent stream, such as a recycled CO₂ stream derived from the fuel gas produced by the gasifier, can also be passed into the gasifier. Alternatively, if sufficient steam is introduced, it can serve as the additional diluent. If at least a portion of the diluent is selected based on a consideration other than facilitating the gasifying and/or reforming reaction in the gasifier, the amount of diluent can be selected by any convenient method. For example, the amount of diluent can be selected so that the amount of diluent replaces the weight of N₂ that would be present in the oxygen-containing stream if air was used as the oxygen-containing stream. As another example, the amount of diluent can be selected to allow for replacement of the same BTU value for heat removal that would be available if N₂ was present based on use of air as the oxygen-containing stream. These types of strategy examples can allow essentially the same or a similar temperature profile to be maintained in the gasifier relative to conventional operation.

In other aspects, the oxygen-containing stream introduced into the gasifier can include sufficient N₂ to allow a portion of the gasifier output stream to be used as an input for ammonia synthesis. In such aspects, sufficient gasification and/or reforming of hydrocarbons can be performed so that the molar ratio of H₂ to N₂ in the gasifier output stream is 1.2 or more, or 1.5 or more, such as 1.2 to 2.5, or 1.2 to 2.0, or 1.5 to 2.5, or 1.5 to 2.0. In such aspects, some options for increasing the H₂ content of the gasifier output stream can include performing steam reforming and/or gasification of hydrocarbons in the gasifier, and adding excess steam to assist with shifting CO to CO₂ (and therefore producing H₂) by the water gas shift reaction. In such aspects, an air separation unit can be used to produce an oxygen-containing stream with a reduced content of N₂. The amount of N₂ in the oxygen-containing stream can be any convenient amount that assists with achieving a desired ratio of H₂ to N₂ in the gasifier output stream, preferably 1.5 or more for ammonia production.

In the gasification zone the reaction between the coke and the steam and the oxygen-containing gas produces a hydrogen and carbon monoxide-containing fuel gas and a partially gasified residual coke product. Conditions in the gasifier are selected accordingly to generate these products. Steam, oxygen, and CO₂ rates will depend upon the rate at which cold coke enters from the reactor and to a lesser extent upon the composition of the coke which, in turn will vary according to the composition of the heavy oil feed and the severity of the cracking conditions in the reactor with these being selected according to the feed and the range of liquid products which is required. The fuel gas product from the gasifier may contain entrained coke solids and these are removed by cyclones or other separation techniques in the gasifier section of the unit; cyclones may be internal cyclones in the main gasifier vessel itself or external in a separate, smaller vessel as described below. The fuel gas product is taken out as overhead from the gasifier cyclones. The resulting partly gasified solids are removed from the gasifier and introduced directly into the coking zone of the coking reactor at a level in the dilute phase above the lower dense phase.

Maintaining Operation of Fluidized Coker with Chemical Production Off-Line

One of the difficulties with integration of a fluidized coking system with a chemicals production system/process is that the compositions of the input streams for chemicals production tend to be specialized. As a result, when the chemicals production system/process needs to be taken off-line (such as for maintenance), oftentimes the associated fluidized coker also has to be stopped, as the outputs from the fluidized coker no longer have a readily available destination. This can have further impact on a refinery, as the ability to use the fluidized coker to process the heavy fractions of a feedstock is also lost.

In some aspects, configurations described herein can overcome this difficulty, so that the fluidized coking process can continue to operate when the chemicals production process is not active. In such aspects, the oxygen-containing stream used for the gasifier can be similar to air, so that the amount of additional diluent (other than N₂) added to the gasifier can be reduced or minimized. In particular, using an oxygen-containing stream with an N₂ content similar to air can avoid the need to introduced recycled CO₂ into the gasifier.

In such aspects, N₂ is removed from the synthesis gas or hydrogen-enriched stream after removal of CO₂. During chemical plant operation, the synthesis gas/hydrogen-enriched stream produced by the nitrogen removal process is delivered to a chemical production process, such as a methanol synthesis process or an ammonia synthesis process. The excess nitrogen stream generated by the nitrogen removal process is mixed with a fuel gas or other hydrocarbon stream to produce a low BTU gas. This low energy content gas is then used as a fuel for an additional process, such as being used as a fuel for one or more refinery processes. When the chemical plant is not in operation, the output from the gasifier can be used as a low BTU gas for the additional process(es). By using this type of configuration, the output stream from the gasifier has a suitable destination whether the chemical plant is in operation or not. This can allow the fluidized coker to continue to run, potentially allowing other refinery processes to also operate in a normal manner.

### Reduced Nitrogen Content in Gasifier Fuel Gas

An example of a fluidized coking system with an integrated gasifier is a Flexicoking™ system available from Exxon Mobil Corporation. In some aspects, the integrated process can allow for reduced or minimized production of inorganic nitrogen compounds by using oxygen from an air separation unit as the oxygen source for gasification. Although the amount of nitrogen introduced as a diluent into the gasification will be reduced, minimized, or eliminated, the integrated process can also allow for gasification of coke while reducing, minimizing, or eliminating production of slag or other glass-like substances in the gasifier. This can be achieved, for example, by recycling a portion of the CO₂ generated during gasification back to the gasifier. Additionally or alternately, other diluent compounds such as steam, CO, and/or inorganic compounds (such as inorganic compounds that are non-reactive in the gasifier environment) can be used as well. Examples of compounds that can be produced from the synthesis gas include, but are not limited to, methanol, ammonia, urea, and fertilizer.

One of the difficulties with using petroleum coke, coal, and/or heavy oils as a feed for gasification is that such feeds can potentially contain a relatively high percentage of transition metals, such as iron, nickel, and vanadium. During conventional operation of a gasifier, these transition metals are converted into a “slag” that tends to be corrosive for the internal structures of the gasifier. As a result, gasifiers can typically have relatively short operating lengths between shutdown events, such as operating lengths of roughly 3 months to 18 months.

For an independently operated gasifier, frequent shutdown events may be acceptable. However, for a gasifier that is integrated to provide heat balance to another process, such as a fluidized bed coker, a short cycle length for the gasifier can force a short cycle length for the coker as well. In order to overcome this problem, a gasifier that is thermally integrated with a fluidized bed coking process, such as a Flexicoking™ process, can be operated under conditions that reduce, minimize, or eliminate formation of slag. Typically this can be achieved by using air as at least a major portion of the oxygen source for the gasifier that is integrated with the fluidized bed coking process. The additional nitrogen in air can provide a diluent for the gasifier environment that can reduce or minimize slag formation. Instead of forming a slag or other glassy type product containing metals, the metals in the coke can be retained in coke form and purged from the integrated system. This can allow the removal/disposition of the metals to be performed in a secondary device or location. By avoiding formation of the corrosive slag, the cycle length of the integrated coker and gasifier can be substantially improved.

One difficulty with operating an integrated coker and gasifier to avoid slag formation is that the resulting fuel gas generated in the gasifier can have a relatively low BTU value. Because of the substantial amount of nitrogen introduced into the gasifier along with the oxygen, the nitrogen content of the fuel gas generated from an integrated fluidized bed/gasifier system can be up to ˜55 vol %. This can present a variety of problems when attempting to find a high value use for the carbon in the fuel gas. For example, this low BTU gas includes a sufficient amount of diluent (such as nitrogen) that it is not directly suitable as a fuel in various types of burners in a refinery setting. Instead, use of the fuel gas as a fuel may require distribution of the fuel gas across multiple burners, so that the fuel gas can be blended with other fuels having a higher energy density. Another difficulty is that the low BTU gas is also a low pressure stream when it emerges from the gasifier. Attempting to compress the fuel gas to match pressures in another processing environment would require compressing the nitrogen in the fuel gas, meaning a substantial additional compression cost with little value in return. However, because the elevated levels of nitrogen make such a fuel gas generally undesirable and/or costly to use, such fuel gas is conventionally burned for heating value. Because this fuel gas is derived from coke that is processed in the gasifier, the net effect of burning this fuel gas is to convert a significant portion of the carbon (typically 20-40%) entering the coker into CO₂ that is released into the atmosphere. In various aspects, the systems and methods described herein can be beneficial for reducing or minimizing the amount of CO₂ that is exhausted into the atmosphere from a fluidized coking/gasifier system.

In various aspects, one or more of the above difficulties related to generation of a low BTU fuel gas from gasification in an integrated coker/gasifier can be overcome by modifying the oxygen source for the gasifier. Instead of using air as the oxygen source, an oxygen-containing stream can be generated by an air separation unit. An air separation unit can provide an oxygen stream with an oxygen content of 96 vol % or more. If desired, the air separation unit can be operated to generate a lower purity oxygen stream and/or additional nitrogen can be added to the oxygen stream so that the oxygen stream used for gasification can include 55 vol % or more of O₂. Thus, use of oxygen from an air separation unit as the oxygen source for a gasifier can reduce, minimize, and/or essentially eliminate the nitrogen content in the gasifier. By avoiding the introduction of substantial amounts of nitrogen into the gasifier, the nitrogen content of the fuel gas can also be reduced to a few percent or less. In various aspects, reducing the nitrogen introduced into the gasifier can allow the combined net volume (or volume percentage) of H₂ and CO in the gas phase product from the gasifier to be greater than 70% of the volume (or volume percentage) of N₂ in the gas phase product, or greater than 100% of the volume of the N₂, or greater than 140 vol % of the N₂, such as up to having substantially no N₂ in the gas phase product.

While reducing the nitrogen content of the fuel gas can be beneficial, the nitrogen introduced into the gasifier also provided a benefit in the form of reducing or minimizing formation of slag or other glassy compounds in the gasifier. In order to maintain a reduced or minimized level of slag formation (such as no slag formation), an alternative diluent can instead be introduced into the gasifier. In various aspects, the alternative diluent can correspond to CO₂, steam, other inorganic compounds, or a combination thereof. Optionally, at least a portion of the alternative diluent can correspond to a recycle stream. Although gasification is typically performed under conditions with a limited amount of oxygen present in the reaction environment, at least some CO₂ is typically formed by the gasification reaction. Additionally, the water-gas shift equilibrium for syngas can potentially favor additional formation of CO₂, depending on the temperature and the relative concentrations of H₂, H₂O, CO, and CO₂. As a result, the fuel gas formed in the gasifier can include a substantial portion of CO₂. This CO₂ formed in the gasifier environment can be separated out by any convenient method, such as by use of a monoethanol amine wash or another type of amine wash. Conveniently, an amine wash can also be suitable for removal of any H₂S that is formed during gasification (such as by reaction of H₂ with sulfur that is present in the coke). In some aspects, multiple amine regeneration steps can be used to desorb CO₂ and H₂S rich streams separately, thus allowing for control over the amount of recycled CO₂ while also allowing for separate handling of H₂S. In some aspects, H₂S can be first removed using selective amine washing, such as a Flexsorb™ process, before using a more general amine wash for CO₂ separation. The pressure at which amine absorption of CO₂ takes place can be in the range of roughly 20 Psia to 1500 Psia (˜140 kPa-a to 10.5 MPa-a) and it is optimized based on the overall configuration of the plant, including factors such as utilization of low pressure or high pressure CO shift reaction section and compression costs. At higher pressures the choice of amine or solvent for absorption of CO₂ expands, which can minimize cost and energy requirement of CO₂ absorption and desorption. At lower pressures amines like methylethylamine (MEA) can be preferred. At moderate pressures amines like methyldiethylamine (MDEA) can be preferred. At high pressures chemical solvents such as methanol can be preferred.

After separation of CO₂ and/or H₂S from the fuel gas, a portion of the CO₂ can be recycled back to the gasifier as a diluent to reduce or minimize formation of slag. In some aspects, the net concentration of O₂ in the oxygen stream introduced into the gasifier, after addition of any diluent and/or steam, can be 22 vol % to 60 vol % relative to the weight of the combined oxygen stream plus diluent and/or steam. In aspects where CO₂ is recycled, at least a portion of the H₂S present in a CO₂ stream can be removed prior to recycling the CO₂ stream to the gasifier. This can assist with maintaining conditions in the gasifier that allow the metals and/or ash content of coke to be removed from the gasifier as part of a coke purge, as opposed to forming a corrosive slag. Alternatively, a portion of the fuel gas after or before a H₂S adsorption (such as a Flexsorb unit) can be compressed and recycled back as the diluent stream.

By reducing or minimizing the content of N₂ in the fuel gas while also reducing or minimizing slag formation, the fuel gas generated by an integrated coker/gasifier can have a substantially increased content of synthesis gas. After removal of sulfur contaminants, water, and/or a majority of CO₂, the resulting fuel gas can correspond to 70 vol % to 99 vol % of H₂ and CO, or 80 vol % to 95 vol %, which are the components of synthesis gas for methanol production. This is a sufficient purity and/or a sufficiently high quality to potentially be valuable to use in synthesis of other compounds. For example, after optional exposure to a water gas-shift catalyst and/or addition of H₂, the synthesis gas can be used as a feed for methanol production.

In addition to methanol production, the type of configuration describe above can also be beneficial for ammonia production. The air separation unit used to generate the oxygen stream for gasification can also produce a high purity nitrogen stream. This high purity nitrogen stream can be combined with a hydrogen stream for ammonia production. In some aspects, the hydrogen can correspond to hydrogen from the synthesis gas generated by gasification. In some aspects, a separate H₂ source can be used to provide hydrogen for ammonia generation. In some aspects, a sufficient portion of N₂ can be left in the O₂ stream used for the gasifier so that the gasifier gas feeding an ammonia plant can also contain at least a major portion of the N₂ needed for ammonia production. For example, the amount of N₂ in the O₂ stream can be selected based on the amount of hydrogen available for ammonia production in the ammonia plant, or (if excess hydrogen is available) the amount of N₂ in the O₂ stream can be selected to provide a desired amount of ammonia production.

Fluidized Coking with Integrated Gasification

In this description, the term “Flexicoking” (trademark of ExxonMobil Research and Engineering Company) is used to designate a fluid coking process in which heavy petroleum feeds are subjected to thermal cracking in a fluidized bed of heated solid particles to produce hydrocarbons of lower molecular weight and boiling point along with coke as a by-product which is deposited on the solid particles in the fluidized bed. The resulting coke can then be converted to a fuel gas by contact at elevated temperature with steam and an oxygen-containing gas in a gasification reactor (gasifier). This type of configuration can more generally be referred to as an integration of fluidized bed coking with gasification.

In various aspects, an integrated fluidized bed coker and gasifier, optionally also including a heater, can be used to process a feed by first coking the feed and then gasifying the resulting coke. This can generate a fuel gas product (withdrawn from the gasifier or the optional heater) that can then be further processed to increase the concentration of synthesis gas in the product. The product with increased synthesis gas concentration can then be used as an input for production of methanol, optionally after further processing to adjust the H₂ to CO ratio in the synthesis gas.

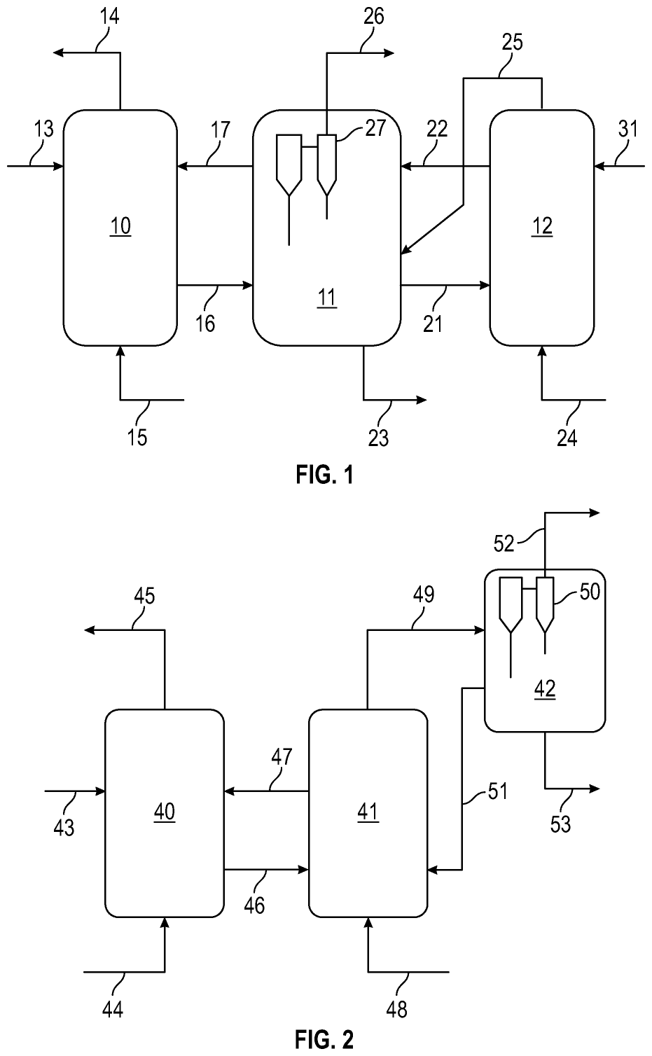

FIG. 1 shows an example of a Flexicoker unit (i.e., a system including a gasifier that is thermally integrated with a fluidized bed coker) with three reaction vessels: reactor, heater and gasifier. The unit comprises reactor section 10 with the coking zone and its associated stripping and scrubbing sections (not separately indicated), heater section 11 and gasifier section 12. The relationship of the coking zone, scrubbing zone and stripping zone in the reactor section is shown, for example, in U.S. Pat. No. 5,472,596, to which reference is made for a description of the Flexicoking unit and its reactor section. A heavy oil feed is introduced into the unit by line 13 and cracked hydrocarbon product withdrawn through line 14. Fluidizing and stripping steam is supplied by line 15. Cold coke is taken out from the stripping section at the base of reactor 10 by means of line 16 and passed to heater 11. The term “cold” as applied to the temperature of the withdrawn coke is, of course, decidedly relative since it is well above ambient at the operating temperature of the stripping section. Hot coke is circulated from heater 11 to reactor 10 through line 17. Coke from heater 11 is transferred to gasifier 12 through line 21 and hot, partly gasified particles of coke are circulated from the gasifier back to the heater through line 22. The excess coke is withdrawn from the heater 11 by way of line 23. In conventional configurations, gasifier 12 is provided with its supply of steam and air by line 24 and hot fuel gas is taken from the gasifier to the heater though line 25. In various aspects, instead of supplying air via a line 24 to the gasifier 12, a stream of oxygen with 55 vol % purity or more can be provided, such as an oxygen stream from an air separation unit. In such aspects, in addition to supplying a stream of oxygen, a stream of an additional diluent gas can be supplied by line 31. The additional diluent gas can correspond to, for example, CO₂ separated from the fuel gas generated during the gasification. The fuel gas is taken out from the unit through line 26 on the heater; coke fines are removed from the fuel gas in heater cyclone system 27 comprising serially connected primary and secondary cyclones with diplegs which return the separated fines to the fluid bed in the heater. The fuel gas from line 26 can then undergo further processing for separation of CO₂ (and/or H₂S) and conversion of synthesis gas to methanol.

It is noted that in some optional aspects, heater cyclone system 27 can be located in a separate vessel (not shown) rather than in heater 11. In such aspects, line 26 can withdraw the fuel gas from the separate vessel, and the line 23 for purging excess coke can correspond to a line transporting coke fines away from the separate vessel. These coke fines and/or other partially gasified coke particles that are vented from the heater (or the gasifier) can have an increased content of metals relative to the feedstock. For example, the weight percentage of metals in the coke particles vented from the system (relative to the weight of the vented particles) can be greater than the weight percent of metals in the feedstock (relative to the weight of the feedstock). In other words, the metals from the feedstock are concentrated in the vented coke particles. Since the gasifier conditions do not create slag, the vented coke particles correspond to the mechanism for removal of metals from the coker/gasifier environment. In some aspects, the metals can correspond to a combination of nickel, vanadium, and/or iron. Additionally or alternately, the gasifier conditions can cause substantially no deposition of metal oxides on the interior walls of the gasifier, such as deposition of less than 0.1 wt % of the metals present in the feedstock introduced into the coker/gasifier system, or less than 0.01 wt %.

In configurations such as FIG. 1, the system elements shown in the figure can be characterized based on fluid communication between the elements. For example, reactor section 10 is in direct fluid communication with heater 11. Reactor section 10 is also in indirect fluid communication with gasifier 12 via heater 11.

As an alternative, integration of a fluidized bed coker with a gasifier can also be accomplished without the use of an intermediate heater. In such alternative aspects, the cold coke from the reactor can be transferred directly to the gasifier. This transfer, in almost all cases, will be unequivocally direct with one end of the tubular transfer line connected to the coke outlet of the reactor and its other end connected to the coke inlet of the gasifier with no intervening reaction vessel, i.e. heater. The presence of devices other than the heater is not however to be excluded, e.g. inlets for lift gas etc. Similarly, while the hot, partly gasified coke particles from the gasifier are returned directly from the gasifier to the reactor this signifies only that there is to be no intervening heater as in the conventional three-vessel Flexicoker™ but that other devices may be present between the gasifier and the reactor, e.g. gas lift inlets and outlets.

FIG. 2 shows an example of integration of a fluidized bed coker with a gasifier but without a separate heater vessel. In the configuration shown in FIG. 2, the cyclones for separating fuel gas from catalyst fines are located in a separate vessel. In other aspects, the cyclones can be included in gasifier vessel 41.

In the configuration shown in FIG. 2, the configuration includes a reactor 40, a main gasifier vessel 41 and a separator 42. The heavy oil feed is introduced into reactor 40 through line 43 and fluidizing/stripping gas through line 44; cracked hydrocarbon products are taken out through line 45. Cold, stripped coke is routed directly from reactor 40 to gasifier 41 by way of line 46 and hot coke returned to the reactor in line 47. Steam and oxygen are supplied through line 48. The flow of gas containing coke fines is routed to separator vessel 42 through line 49 which is connected to a gas outlet of the main gasifier vessel 41. The fines are separated from the gas flow in cyclone system 50 comprising serially connected primary and secondary cyclones with diplegs which return the separated fines to the separator vessel. The separated fines are then returned to the main gasifier vessel through return line 51 and the fuel gas product taken out by way of line 52. Coke is purged from the separator through line 53. The fuel gas from line 52 can then undergo further processing for separation of CO₂ (and/or H₂S) and conversion of synthesis gas to methanol.

The coker and gasifier can be operated according to the parameters necessary for the required coking processes. Thus, the heavy oil feed will typically be a heavy (high boiling) reduced petroleum crude; petroleum atmospheric distillation bottoms; petroleum vacuum distillation bottoms, or residuum; pitch; asphalt; bitumen; other heavy hydrocarbon residues; tar sand oil; shale oil; or even a coal slurry or coal liquefaction product such as coal liquefaction bottoms. Such feeds will typically have a Conradson Carbon Residue (ASTM D189-165) of at least 5 wt. %, generally from 5 to 50 wt. %. Preferably, the feed is a petroleum vacuum residuum.

A typical petroleum chargestock suitable for processing in a fluidized bed coker can have a composition and properties within the ranges set forth below.

TABLE 1
Example of Coker Feedstock

| | | |
|---|---|---|
| Conradson Carbon | 5 to 40 | wt. % |
| API Gravity | −10 to 35° | |
| Boiling Point | 340° C.+ to 650° C.+ | |
| Sulfur | 1.5 to 8 | wt. % |
| Hydrogen | 9 to 11 | wt. % |
| Nitrogen | 0.2 to 2 | wt. % |
| Carbon | 80 to 86 | wt. % |
| Metals | 1 to 2000 | wppm |

More generally, the feed to the fluidized bed coker can have a T10 distillation point of 343° C. or more, or 371° C. or more.

The heavy oil feed, pre-heated to a temperature at which it is flowable and pumpable, is introduced into the coking reactor towards the top of the reactor vessel through injection nozzles which are constructed to produce a spray of the feed into the bed of fluidized coke particles in the vessel. Temperatures in the coking zone of the reactor are typically in the range of 450° C. to 850° C. and pressures are kept at a relatively low level, typically in the range of 120 kPag to 400 kPag (17 psig to 58 psig), and most usually from 200 kPag to 350 kPag (29 psig to 51 psig), in order to facilitate fast drying of the coke particles, preventing the formation of sticky, adherent high molecular weight hydrocarbon deposits on the particles which could lead to reactor fouling. The conditions can be selected so that a desired amount of conversion of the feedstock occurs in the fluidized bed reactor. The coking reaction and the amount of conversion can be selected to be similar to the values used in a conventional fluidized coking reaction. For example, the conditions can be selected to achieve at least 10 wt % conversion relative to 343° C. (or 371° C.), or at least 20 wt % conversion relative 343° C. (or 371° C.), or at least 40 wt % conversion relative to 343° C. (or 371° C.), such as up to 80 wt % conversion or possibly still higher. The light hydrocarbon products of the coking (thermal cracking) reactions vaporize, mix with the fluidizing steam and pass upwardly through the dense phase of the fluidized bed into a dilute phase zone above the dense fluidized bed of coke particles. This mixture of vaporized hydrocarbon products formed in the coking reactions flows upwardly through the dilute phase with the steam at superficial velocities of ˜1 to 2 meters per second (˜3 to 6 feet per second), entraining some fine solid particles of coke which are separated from the cracking vapors in the reactor cyclones as described above. The cracked hydrocarbon vapors pass out of the cyclones into the scrubbing section of the reactor and then to product fractionation and recovery. The cracked hydrocarbon vapors can include one or more liquid products with a boiling range of 343° C. or less. Examples of 343° C.− liquid products include coker naphtha and coker gas oil.

As the cracking process proceeds in the reactor, the coke particles pass downwardly through the coking zone, through the stripping zone, where occluded hydrocarbons are stripped off by the ascending current of fluidizing gas (steam). They then exit the coking reactor and pass to the gasification reactor (gasifier) which contains a fluidized bed of solid particles and which operates at a temperature higher than that of the reactor coking zone. In the gasifier, the coke particles are converted by reaction at the elevated temperature with steam and an oxygen-containing gas into a fuel gas comprising carbon monoxide and hydrogen.

The gasification zone is typically maintained at a high temperature ranging from 850° C. to 1000° C. (1560° F. to 1830° F.) and a pressure ranging from 0 kPag to 1000 kPag (0 psig to 150 psig), preferably from 200 kPag to 400 kPag (30 psig to 60 psig). Steam and an oxygen-containing gas having a low nitrogen content, such as oxygen from an air separation unit or another oxygen stream including 95 vol % or more of oxygen, or 98 vol % or more, are passed into the gasifier for reaction with the solid particles comprising coke deposited on them in the coking zone. A separate diluent stream, such as a recycled CO₂ or H₂S stream derived from the fuel gas produced by the gasifier, can also be passed into the gasifier. The amount of diluent can be selected by any convenient method. For example, the amount of diluent can be selected so that the amount of diluent replaces the weight of N₂ that would be present in the oxygen-containing stream if air was used as the oxygen-containing stream. As another example, the amount of diluent can be selected to allow for replacement of the same BTU value for heat removal that would be available if N₂ was present based on use of air as the oxygen-containing stream. These types of strategy examples can allow essentially the same or a similar temperature profile to be maintained in the gasifier relative to conventional operation.

In the gasification zone the reaction between the coke and the steam and the oxygen-containing gas produces a hydrogen and carbon monoxide-containing fuel gas and a partially gasified residual coke product. Conditions in the gasifier are selected accordingly to generate these products. Steam, oxygen, and CO₂ rates will depend upon the rate at which cold coke enters from the reactor and to a lesser extent upon the composition of the coke which, in turn will vary according to the composition of the heavy oil feed and the severity of the cracking conditions in the reactor with these being selected according to the feed and the range of liquid products which is required. The fuel gas product from the gasifier may contain entrained coke solids and these are removed by cyclones or other separation techniques in the gasifier section of the unit; cyclones may be internal cyclones in the main gasifier vessel itself or external in a separate, smaller vessel as described below. The fuel gas product is taken out as overhead from the gasifier cyclones. The resulting partly gasified solids are removed from the gasifier and introduced directly into the coking zone of the coking reactor at a level in the dilute phase above the lower dense phase.

### Methanol Production

After withdrawing the fuel gas from the heater or gasifier, the fuel gas can undergo further processing to produce a stream with an increased concentration of CO and H₂. Because a reduced or minimized amount of nitrogen was introduced into the gasifier as part of the oxygen stream, the amount of nitrogen in the fuel gas can also be minimal, such as 5 vol % or less. At this level, the nitrogen can be passed into a methanol synthesis process without requiring separation.

Other gases present in the fuel gas can be separated to improve the subsequent methanol synthesis process. For example, as noted above, the gasification conditions can result in formation of a substantial amount of CO₂, corresponding to 5 vol % to 20 vol % of the fuel gas. This CO₂ can be removed from the fuel gas by any convenient method. Suitable methods for separation of CO₂ from the fuel gas can include, but are not limited to, amine washing and cryogenic separation. After separation of the CO₂ from the fuel gas, the CO₂ can be recovered (if necessary) and then used as in any convenient manner. In some aspects, at least a portion of the CO₂ can be used as a diluent for the gasification process. As discussed further below, CO₂ can potentially be converted to methanol under the methanol synthesis conditions, so complete removal of CO₂ is not necessary.

Another gas present in the fuel gas can be H₂S. For many types of heavy petroleum feeds, the feed can include a substantial amount of sulfur. This sulfur can be incorporated into the coke and then converted to H₂S in the gasifier. Any convenient method for removal of H₂S can be used. In aspects where an amine wash is used for CO₂ separation, the amine wash can also be effective for H₂S removal.

During methanol synthesis, carbon monoxide and hydrogen can react over a catalyst to produce methanol. Commercial methanol synthesis catalysts can be highly selective, with selectivities of greater than 99.8% possible under optimized reaction conditions. Typical reaction conditions can include pressures of 5 MPa to 10 MPa and temperatures of 250° C. to 300° C. With regard to the syngas input for methanol synthesis, the preferred ratio of H₂ to CO (˜2:1 H₂:CO) does not match the typical ratio generated by a gasifier. For example, a typical Flexicoking™ H₂:CO ratio is ˜1:1. In some aspects, production of methanol using syngas from a gasifier can be improved by addition of H₂ to the syngas. Additionally or alternately, catalysts that facilitate methanol formation from syngas can sometimes additionally facilitate the water-gas shift reaction. As a result, the reaction scheme below shows that CO₂ can also be used to form methanol:

2H₂+CO=>CH₃OH

3H₂+CO₂=>CH₃OH+H₂O

For methanol synthesis reactions, the composition of the synthesis gas input can be characterized by the Module value M:

M=[H₂—CO₂]/[CO+CO₂]

Module values close to 2 can generally be suitable for production of methanol, such as values of M that are at least 1.7, or at least 1.8, or at least 1.9, and/or less than 2.3, or less than 2.2, or less than 2.1. As can be noted from the Module Value equation above, in addition to the ratio of H₂ to CO, the ratio of CO to CO₂ in the syngas can impact the reaction rate of the methanol synthesis reaction.

The output stream from a gasifier can contain relatively high concentrations of H₂, CO, CO₂, and water. Through a combination of separations, (reverse) water gas shift reactions, and/or other convenient mechanisms, the composition of the fuel gas from the gasifier and/or a stream derived/withdrawn from the fuel gas can be adjusted. The adjustment of the composition can include removing excess water and/or CO₂, adjusting the ratio of H₂:CO, adjusting the Module value M, or a combination thereof. For example, a typical fuel gas from the gasifier may have an H₂:CO ratio of ˜1:1. Removal of CO₂ from the fuel gas can facilitate a subsequent water gas shift reaction to increase this ratio to closer to 2:1 and/or to increase the Module value M of the stream to closer to 2.

In a typical methanol plant, a large percentage of the reactor exhaust can be recycled after recovery of methanol liquid, due to low conversion per pass. In some configurations, the output from the methanol synthesis reaction can be separated into a liquid alcohol product, a recycle syngas stream, and a vented purge. The vented purge can contain syngas components, fuel components (e.g. methane), and inerts. In some aspects, at least a portion of the vented purge can be used to raise steam for heating the syngas production. Additionally or alternately, at least a portion of the purged gas can be upgraded to syngas in the gasifier of the coker. Further additionally or alternately, the water produced in the methanol plant can be used as wash water in the coker light product recovery section.

### Ammonia Production

Ammonia can typically be made from H₂ and N₂ via the Haber-Bosch process at elevated temperature and pressure. Conventionally, the inputs can be a) purified H₂, which can be made from a multi-step process that can typically require steam methane reforming, water gas shift, water removal, and trace carbon oxide conversion to methane via methanation; and b) purified N₂, which can typically be derived from air via pressure swing adsorption and/or an air separation unit.

Additionally or alternately, the purified H₂ for ammonia production can be provided from the syngas generated by the gasifier (as part of the fuel gas). As described above, the syngas generated by the gasifier can be further processed to remove impurities such as sulfur. For ammonia synthesis, the hydrogen stream can preferably be substantially free of impurities such as H₂S. If a portion of the syngas generated by the gasifier is used as a source of hydrogen for ammonia synthesis, the syngas can first be reacted in a water-gas shift reactor to maximize the amount of H₂ relative to CO. Water-gas shift is a well-known reaction, and typically can be done at “high” temperatures (from ˜300° C. to ˜500° C.) and “low” temperatures (from ˜100° C. to ˜300° C.) with the higher temperature catalyst giving faster reaction rates, but with higher exit CO content, followed by the low temperature reactor to further shift the syngas to higher H₂ concentrations. Following this, the gas can undergo separation via one or more processes to purify the H₂. This can involve, for example, condensation of the water, removal of CO₂, purification of the H₂ and then a final methanation step at elevated pressure (typically 15 barg to 30 barg, or 1.5 MPag to 3 MPag) to ensure that as many carbon oxides as possible can be eliminated. Lastly, the H₂ stream can be compressed to ammonia synthesis conditions of roughly 60 barg (˜6 MPag) to 180 barg (18 MPag). Typical ammonia processes can be performed at 350° C. to 500° C., such as at 450° C. or less, and can result in low conversion per pass (typically less than 20%) and a large recycle stream.

In some aspects, the gasification CO₂ recirculation system described herein can also incorporate a purge CO₂ stream to reduce or minimize the need for CO₂ separation or destruction at high pressure before the ammonia plant. In some aspects, the purge stream from the ammonia plant can be recycled to gasifier for additional recovery of synthesis gas.

Urea is another large chemical product that can be made by the reaction of ammonia with CO₂. The basic process, developed in 1922, is also called the Bosch-Meiser urea process after its discoverers. The various urea processes can be characterized by the conditions under which urea formation takes place and the way in which unconverted reactants are further processed. The process can consist of two main equilibrium reactions, with incomplete conversion of the reactants. The net heat balance for the reactions can be exothermic. The first equilibrium reaction can be an exothermic reaction of liquid ammonia with dry ice (solid CO₂) to form ammonium carbamate (H₂N—COONH₄):

2NH₃+CO₂ [symbol] H₂N—COONH₄

The second equilibrium reaction can be an endothermic decomposition of ammonium carbamate into urea and water:

H₂N-COONH₄ [symbol] (NH₂)₂CO+H₂O

The urea process can use liquefied ammonia and CO₂ at high pressure as process inputs. In prior art processes, carbon dioxide is typically provided from an external resource where it must be compressed to high pressure. In contrast, the current process, as shown in FIG. 6, can produce a high pressure carbon dioxide stream suitable for reaction with the liquid ammonia product from the ammonia synthesis reaction. It is noted that the gasification O₂ input can be varied to adjust the amount of CO₂ produced. In addition, CO produced in the gasification step and steam can be reacted to produce more H₂ and CO₂ for NH₃ and increased urea production.

In various aspects, the urea process can be integrated into a combined system with an ammonia synthesis process and a Flexicoker™ type process (i.e., fluidized bed coker including an integrated gasifier). This integrated approach can reduce and/or eliminate many processes from the conventional approach, which can require an ammonia plant (steam reformer, water gas shift, pressure swing adsorption to produce H₂+air separation plant) plus a separate supply of CO₂ typically made remotely and then transported to the plant. The current system can eliminate many of these processes, as well as providing CO₂ for use in forming the urea. Specifically, rather than transport CO₂ as dry ice for use at a remote urea plant, carbon dioxide can be provided from separation of the syngas stream from the gasifier.

### Configuration Example: Modification of Operation of Gasifier for Production of Synthesis Gas

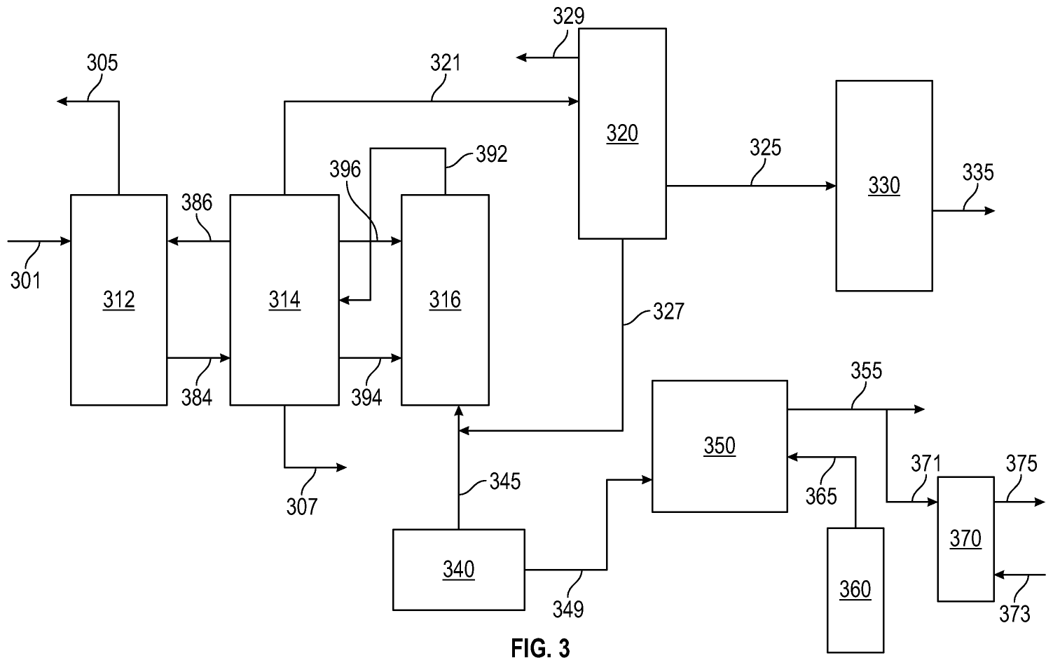

FIG. 3 shows an example of a configuration that provides an integrated fluidized bed coker and gasifier, along with optional methanol synthesis, ammonia synthesis, and urea synthesis processes. It is noted that any convenient combination of the methanol synthesis, ammonia synthesis, and urea synthesis processes can be present independently from each other. To the degree that an output of one optional process (such as ammonia) is described as being an input for a second optional process (such as urea synthesis), it is understood that in some aspects, the input for the second optional process can be derived from another conventional source.

In FIG. 3, a feed 301 suitable for coking is introduced into fluidized bed coker 312. The feed 301 can correspond to a heavy oil feed, or any other convenient feed typically used as an input for a coker. In the configuration shown in FIG. 3, the fluidized bed coker 312 is integrated with a heater 314 and a gasifier 316. This combination of elements is similar to the configuration shown in FIG. 1.

In FIG. 3, fluidized bed coker 312 generates a primary product 305 that includes fuel boiling range liquids generated during the coking process. Heat for coker 312 is provided by hot coke recycle line 386, while cold coke from coker 312 is passed into heater 314 via line 384. Coke from heater 314 is transferred to gasifier 316 through line 394 and hot, partly gasified particles of coke are circulated from the gasifier back to the heater through line 396. Fuel gas generated in gasifier 316 is returned to heater 314 via line 392. It is noted that gasifier 316 does not generate a slag that is separately removed from the gasifier. Instead, excess coke is withdrawn from the heater 314 by way of line 307. It is noted that the steam lines for fluidization of the coke in the fluidized bed and the gasifier are not shown in FIG. 3.

Fuel gas provided from gasifier 316 to heater 314 via line 392 can provide the fluidization needed in heater 314. The fuel gas can be withdrawn from heater 314 via line 321, optionally after passing through cyclone separators (not shown) for removal of coke fines from the fuel gas. The fuel gas in line 321 can be passed into a separation stage 320 for separation of CO₂ from the fuel gas. A portion of the CO₂ can be vented and/or withdrawn via line 329 for use in any convenient manner. Another portion of the CO₂ 327 can be used a recycle stream and returned to gasifier 316. In the configuration shown in FIG. 3, this is accomplished by combining the portion of the CO₂ 327 with oxygen 345 from air separation unit 340. The combined oxygen 345 and CO₂ 327 are then passed into gasifier 316. Optionally, separation stage 320 can also be used for removal of H₂S from the fuel gas stream 321. Optionally, one or more additional separation stages may be present if removal of any other impurities from fuel gas stream 321 is desired. After separation of CO₂ (and/or other impurities), the remaining portion of the fuel gas stream can correspond to a synthesis gas stream 325. The synthesis gas stream 325 can be passed into a methanol synthesis plant 330 for production of methanol 335.

In addition to providing a high purity oxygen stream 345 to gasifier 316, the air separation unit 340 can also generate a nitrogen stream 349 that has a nitrogen content of 95 vol % or more. This can be passed into an ammonia synthesis process 350. The ammonia synthesis process 350 can also receive a hydrogen stream 365 corresponding to 98 vol % or more of hydrogen. In FIG. 3, hydrogen stream 365 is provided from a hydrogen source 360. Optionally, hydrogen stream 365 can be derived at least in part from synthesis gas stream 325. The hydrogen stream 365 and nitrogen stream 349 can be reacted in ammonia synthesis process 350 to form ammonia output 355. Optionally, a portion 371 of ammonia output 355 can be passed into a urea synthesis process 370 for production of a urea stream 375. The urea synthesis process 370 can also require a stream of CO₂ 373. Optionally, at least a portion of CO₂ stream 373 can correspond to CO₂ derived from CO₂ vent and/or withdrawal stream 329.

### Configuration Example: Modification of Processing of Gasifier Fuel Gas for Carbon Capture and Sulfur Removal

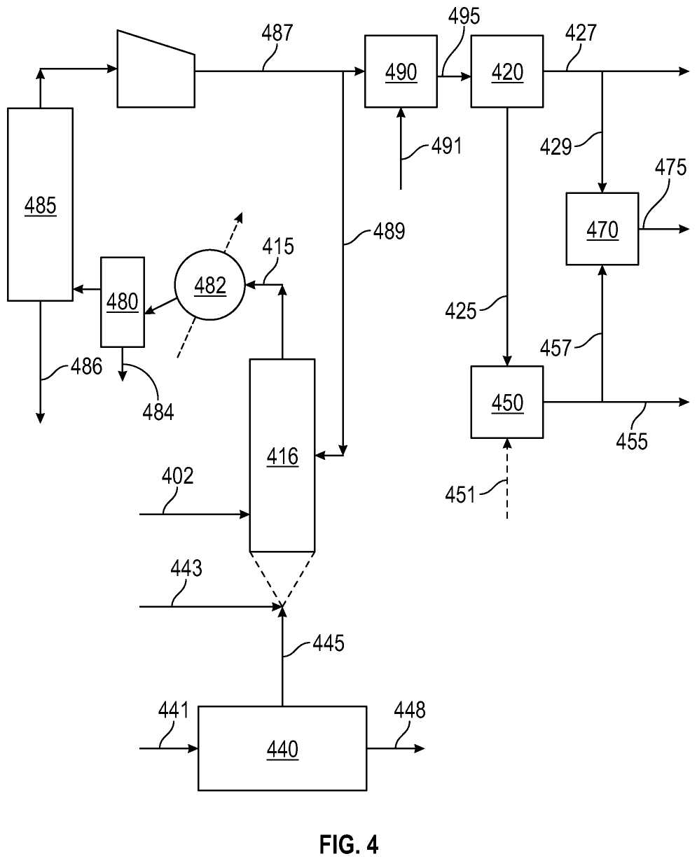

FIG. 4 shows an example of a configuration that provides an integrated fluidized bed coker and gasifier, along with sulfur removal, carbon capture, and optional ammonia synthesis and/or urea synthesis processes. It is noted that any convenient combination of the ammonia synthesis and urea synthesis processes can be present independently from each other. To the degree that an output of one optional process (such as ammonia) is described as being an input for a second optional process (such as urea synthesis), it is understood that in some aspects, the input for the second optional process can be derived from another conventional source.

In the example shown in FIG. 4, the fluidized coker and any optional heater are not shown. Instead, FIG. 4 is focused on the configuration surrounding the gasifier 416.

In FIG. 4, cold coke (not shown) is passed into gasifier 416 for partial combustion of the coke. Hot coke particles are provided (not shown) for return to the coker and/or optional heater of the fluidized coking system. In addition to coke particles, gasifier 416 also receives a hydrocarbon-containing stream 402. Hydrocarbon-containing stream 402 can correspond to methane, natural gas, fuel gas, and/or another convenient stream including hydrocarbons that are suitable for reforming and/or gasification in the gasifier in order to produce additional H₂. In the example shown in FIG. 4, hydrocarbon-stream 402 is introduced into an intermediate part of gasifier 416. Gasifier 416 further receives an oxygen-containing stream 445, such as an oxygen-containing stream generated by separating air 441 in an air separation unit 440. The air separation unit 440 can also generate a N₂ rich purge 448. Alternatively, a membrane separator or swing adsorber (not shown) could be used to generate a stream enriched in O₂ relative to N₂ (as compared to air). Gasifier 416 also receives steam 443. In FIG. 4, oxygen-containing stream 445 and steam 443 are shown as being introduced as a fluidizing gas at the bottom (i.e., a lower zone) of gasifier 416, but other convenient methods of introducing the oxygen-containing stream 445 and steam 443 can also be used. The gasifier generates a fuel gas product 415. In the configuration shown in FIG. 4, gasifier 416 does not generate a slag that is separately removed from the gasifier.

The fuel gas 415 is processed in several steps to form a desired synthesis gas intermediate or final product while also removing sulfur and capturing CO₂. In the example shown in FIG. 4, fuel gas 415 is cooled 482 prior to passing through a knock-out separation stage 480 for removal of water and particle fines 484. This can include passing the fuel gas 415 through cyclone separators (not shown). The effluent from knock-out separation stage 480 is then passed into a sulfur removal stage 485. The sulfur removal stage 485 can correspond to an adsorbent stage, such as a Flexsorb™ sulfur removal stage. Preferably, the sulfur removal stage 485 is selective for removal of sulfur 486 (such as in the form of H₂S) while reducing or minimizing removal of CO₂. A portion of the resulting desulfurized effluent 487 can optionally be used as an additional diluent stream 489 for the gasifier. The remainder of desulfurized effluent 487 can then be passed into a water gas shift stage 490. In addition, steam 491 is added to the water gas shift stage, to assist with further creation of H₂. The shifted desulfurized effluent 495 can then be passed into CO₂ separation stage 420. Alternatively, stages 490 and 420 can be located before the compression section or after an additional stage of compression. The pressure to conduct these stages is determined by the particular site costs and economics. Any convenient type of CO₂ separation can be used, such as cryogenic separation, membrane separation, and/or adsorption (including swing adsorption). The resulting high purity CO₂ 427 can then be sequestered. Optionally, a portion 429 of the CO₂ can be used for chemical production.

CO₂ separation stage 420 also generates a stream 425 enriched in H₂ and/or enriched in synthesis gas (H₂+CO) and/or enriched in H₂ and N₂. This H₂ enriched stream 425 can then be used for chemical production. In the example shown in FIG. 4, the H₂ enriched stream 425 is passed into ammonia synthesis process 450 to produce ammonia 455. Optionally additional H₂ stream 451 can also be provided to ammonia synthesis process 450. Optionally, a portion 457 of the resulting ammonia 455 can be passed into a urea synthesis process 470, along with portion 429 of CO₂, for production of urea 475.

### Configuration Example: Modification of Processing of Gasifier Fuel Gas for Continuous Operation and Sulfur Consumption

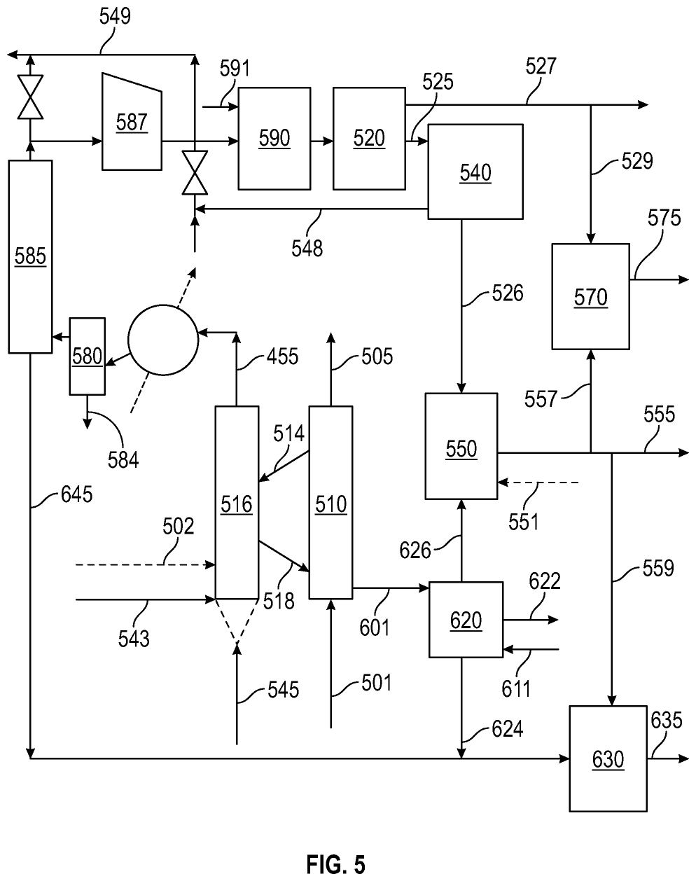

FIG. 5 shows an example of a configuration that provides an integrated fluidized bed coker and gasifier, along with sulfur removal, carbon capture, and optional ammonia synthesis, urea synthesis, and fertilizer synthesis processes. It is noted that any convenient combination of the ammonia synthesis, urea synthesis, and fertilizer synthesis processes can be present independently from each other. To the degree that an output of one optional process (such as ammonia) is described as being an input for a second optional process (such as urea synthesis), it is understood that in some aspects, the input for the second optional process can be derived from another conventional source.

In FIG. 5, a mixed stream of coker feed and steam 501 is passed into fluidized coker 510 to generate coker effluent 505. Cold coke 514 is passed into gasifier 516 for partial combustion of the coke. Hot coke particles are provided 518 for return to the coker 510. Optionally, the fluidized coking system can also include a heater (not shown). The fluidized coker 510 can also generate a sour water stream 601 that includes a mixture of at least water, H₂S, and optionally NH₃. The sour water stream 601 can be passed into sour water processing stage 620 to produce water 622, sulfur product 624, and optionally ammonia product 626. Optionally, sour water 611 from other locations in the refinery can also be processed in sour water processing stage 620.

In addition to coke particles, gasifier 516 can also receive an optional hydrocarbon-containing stream 502. Hydrocarbon-containing stream 502 can correspond to methane, natural gas, fuel gas, and/or another convenient stream including hydrocarbons that are suitable for H₂ generation. Gasifier 516 further receives an oxygen-containing stream 545, such as air. Gasifier 516 also receives steam 543. In FIG. 5, oxygen-containing stream 545 and steam 543 are shown as being introduced as a fluidizing gas at the bottom of gasifier 516, but other convenient methods of introducing the oxygen-containing stream 545 and steam 543 can also be used. The gasifier generates a fuel gas product 515. In the configuration shown in FIG. 5, gasifier 516 does not generate a slag that is separately removed from the gasifier.

The fuel gas 515 is processed in several steps to form a desired synthesis gas intermediate or final product while also removing sulfur and capturing CO₂. In the example shown in FIG. 5, fuel gas 515 is cooled prior to passing through a knock-out separation stage 580 for removal of water and particle fines 584. This can include passing the fuel gas 515 through cyclone separators (not shown). The effluent from knock-out separation stage 580 is then passed into a sulfur removal stage 585. The sulfur removal stage 585 can correspond to an adsorbent stage, such as a Flexsorb™ sulfur removal stage. Preferably, the sulfur removal stage 585 is selective for removal of sulfur while reducing or minimizing removal of CO₂. The sulfur removal stage 585 can generate a sulfur product 645 and a desulfurized effluent 587. The desulfurized effluent 587 can then be passed into a water gas shift stage 590. Optionally, additional steam 591 can be added to the water gas shift stage, to assist with further creation of H₂. The shifted desulfurized effluent can then be passed into CO₂ separation stage 520. Any convenient type of CO₂ separation can be used, such as cryogenic separation, membrane separation, and/or adsorption (including swing adsorption). The resulting high purity CO₂ 527 can then be sequestered. Optionally, a portion 529 of the CO₂ can be used for chemical production.

CO₂ separation stage 520 also generates a stream 525 enriched in H₂ and/or enriched in synthesis gas (H₂+CO) and/or enriched in (H₂+N₂). In the configuration shown in FIG. 5, the stream 525 enriched in H₂ also contains a substantial portion of N₂, since air was used as the oxygen-containing stream 545. At least a portion of the N₂ can be removed using a nitrogen separation stage 540 to generate a stream 526 with a reduced nitrogen content. The nitrogen separation stage 540 can also generate an N₂ rich purge 548. The nitrogen separation stage can correspond to a refrigeration unit, membrane separator, a swing adsorber, or another convenient process unit for selective removal of N₂. During chemical production, at least a portion of the N₂ rich purge 548 can be combined with a fuel 509 to form a low energy content fuel gas 549. This will ensure continuous operations of the special burners for low BTU gas which when the chemical plant is shut down for maintenance or other purposes. The low energy content fuel gas 549 can be burned in the special burners without upsetting the operation of the furnaces.

The stream 526 can then be used for chemical production. In the example shown in FIG. 5, the H₂ enriched stream 526 is passed into ammonia synthesis process 550 for production of ammonia 555. Optionally additional H₂ stream 551 can also be provided to ammonia synthesis process 550. A first portion 557 of the resulting ammonia 555 is passed into a urea synthesis process 570, along with portion 529 of CO₂, for production of urea 575. A second portion 579 of the ammonia can be passed into fertilizer synthesis process 630 to produce a fertilizer product 635. The fertilizer synthesis process 630 can also use sulfur product 645 and/or sulfur product 626.

In the event that the chemical production portion of the system is shut down, the operation of the configuration in FIG. 5 can be altered. Instead of passing desulfurized effluent 587 into the CO₂ separation stage 590, the desulfurized effluent 587 can be used as fuel for one or more refinery processes. This replaces fuel 509, which is not formed when the chemical production portion of the system is shut down.

### Additional Embodiments

### Embodiment 1

A method for producing synthesis gas or products derived from synthesis gas, comprising: exposing a feedstock comprising a T10 distillation point of 343° C. or more to a fluidized bed comprising solid particles in a reactor under thermal cracking conditions to form at least a 343° C.− liquid product, the thermal cracking conditions comprising 10 wt % or more conversion of the feedstock relative to 343° C., the thermal cracking conditions being effective for depositing coke on the solid particles; introducing an oxygen-containing stream, a hydrocarbon-containing stream, and steam into a gasifier, passing at least a portion of the solid particles comprising deposited coke from the reactor to the gasifier, the solid particles optionally comprising coke; exposing the at least a portion of the solid particles comprising deposited coke to gasification conditions in the gasifier to form a gas phase product comprising H₂, CO, and CO₂ and partially gasified coke particles, the gasification conditions further comprising conditions for at least one of reforming and gasifying hydrocarbons in the hydrocarbon-containing stream in the presence of the steam, the gas phase product comprising a combined volume of H₂ and CO that is greater than 70% (or greater than 140%) of a volume of N₂ in the gas phase product; removing at least a first portion of the partially gasified coke particles from the gasifier; and passing at least a second portion of the partially gasified coke particles from the gasifier to the reactor.

### Embodiment 2

The method of Embodiment 1, further comprising separating a stream comprising O₂ and N₂ to form the oxygen-containing stream and a nitrogen-containing stream, the oxygen-containing stream comprising 55 vol % or more of O₂ prior to combining the oxygen-containing stream with at least one of the hydrocarbon-containing stream and the steam.

### Embodiment 3

The method of Embodiment 2, further comprising exposing at least a portion of the nitrogen stream and at least a portion of the gas phase product to a catalyst under ammonia synthesis conditions to form ammonia.

### Embodiment 4

The method of Embodiment 3, further comprising exposing at least a first portion of the ammonia to a urea synthesis catalyst in the presence of CO₂ under urea synthesis conditions to form urea.

### Embodiment 5

The method of Embodiment 4, further comprising exposing at least a portion of the urea to a catalyst in the presence of sulfur to form a fertilizer product.

### Embodiment 6

The method of Embodiment 5, further comprising separating H₂S from the gas phase product to form a desulfurized gas phase product and a sulfur-containing product, and wherein at least a second portion of the ammonia is exposed to a catalyst in the presence of at least a portion of the sulfur-containing product to form the fertilizer product.

### Embodiment 7

The method of Embodiment 6, further comprising separating CO₂ from at least one of the gas phase product and the desulfurized gas phase product to form a synthesis gas stream and a CO₂-containing product.

### Embodiment 8

The method of Embodiment 7, wherein the at least a first portion of the ammonia is exposed to the urea synthesis catalyst in the presence of at least a first portion of the CO₂-containing product to form urea, a second portion of the CO₂-containing product optionally being recycled to the gasifier as an additional diluent.

### Embodiment 9

The method of Embodiment 8, wherein the at least a portion of the gas phase product comprises at least a portion of the synthesis gas stream.

### Embodiment 10

The method of any of the above embodiments, wherein passing at least a portion of the solid particles comprising deposited coke from the reactor to the gasifier comprises passing the at least a portion of the solid particles comprising deposited coke to a heater, and passing the at least a portion of the solid particles comprising deposited coke from the heater to the gasifier.

### Embodiment 11

The method of any of the above embodiments, wherein passing at least a second portion of the partially gasified coke particles from the gasifier to the reactor comprises passing the at least a second portion of partially gasified coke particles to a heater, and passing the at least a second portion of the partially gasified coke particles from the heater to the reactor.

### Embodiment 12

The method of any of the above embodiments, further comprising exposing the gas phase product to water gas shift conditions to increase the combined volume of H₂ and CO.

### Embodiment 13

A method for producing synthesis gas or products derived from synthesis gas, comprising: exposing a feedstock comprising a T10 distillation point of 343° C. or more to a fluidized bed comprising solid particles in a reactor under thermal cracking conditions to form a 343° C.− liquid product, the thermal cracking conditions comprising 10 wt % or more conversion of the feedstock relative to 343° C., the thermal cracking conditions being effective for depositing coke on the solid particles, the solid particles optionally comprising coke; introducing steam and a stream comprising O₂ and N₂ into a gasifier; passing at least a portion of the solid particles comprising deposited coke from the reactor to the gasifier; exposing the at least a portion of the solid particles comprising deposited coke to gasification conditions in the gasifier to form a gas phase product comprising H₂, N₂, CO, and CO₂ and partially gasified coke particles; removing at least a first portion of the partially gasified coke particles from the gasifier, passing at least a second portion of the partially gasified coke particles from the gasifier to the reactor, separating, during a first time period, CO₂ from at least a portion of the gas phase product to form a dilute synthesis gas stream; separating, during the first time period, N₂ from the dilute synthesis gas stream to form a nitrogen-containing stream and a synthesis gas stream; exposing, during the first time period, at least a portion of the synthesis gas stream to a catalyst in a synthesis reactor to form a chemical product, the chemical product optionally comprising at least one of methanol and ammonia; combining, during the first time period, a hydrocarbon-containing stream with the nitrogen-containing stream to form a low-BTU gas, at least a portion of the low-BTU gas being passed as a fuel or reagent to an additional process, the hydrocarbon-containing stream optionally further comprising H₂; stopping, during a second time period, the operation of the synthesis reactor, and passing, during the second time period, at least a portion of the gas phase product to the additional process.

### Embodiment 14

The method of Embodiment 13, further comprising separating, during the first time period, H₂S from the gas phase product to form a desulfurized gas phase product, the CO₂ being separated during the first time period from the desulfurized gas phase product; and separating, during the second time period, H₂S from the gas phase product prior to passing the at least a portion of the gas phase product to the additional process.

### Embodiment 15

The method of Embodiments 13 or 14, further comprising introducing a second hydrocarbon-containing stream into the gasifier, the gasification conditions further comprising conditions for at least one of gasifying and reforming hydrocarbons in the second hydrocarbon-containing stream in the presence of the steam.

When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

The present invention has been described above with reference to numerous embodiments and specific examples. Many variations will suggest themselves to those skilled in this art in light of the above detailed description. All such obvious variations are within the full intended scope of the appended claims.

1. A method for producing synthesis gas or products derived from synthesis gas, comprising:
exposing a feedstock comprising a T10 distillation point of 343° C. or more to a fluidized bed comprising solid particles in a reactor under thermal cracking conditions to form at least a 343° C.− liquid product, the thermal cracking conditions comprising 10 wt % or more conversion of the feedstock relative to 343° C., the thermal cracking conditions being effective for depositing coke on the solid particles;
introducing an oxygen-containing stream, a hydrocarbon-containing stream, and steam into a gasifier;
passing at least a portion of the solid particles comprising deposited coke from the reactor to the gasifier;
exposing the at least a portion of the solid particles comprising deposited coke to gasification conditions in the gasifier to form a gas phase product comprising H₂, CO, and CO₂ and partially gasified coke particles, the gasification conditions further comprising conditions for at least one of reforming and gasifying hydrocarbons in the hydrocarbon-containing stream in the presence of the steam, the gas phase product comprising a combined volume of H₂ and CO that is greater than 70% of a volume of N₂ in the gas phase product;
removing at least a first portion of the partially gasified coke particles from the gasifier; and
passing at least a second portion of the partially gasified coke particles from the gasifier to the reactor.

2. The method of claim 1, further comprising exposing the gas phase product to water gas shift conditions to increase the combined volume of H₂ and CO.

3. The method of claim 1, further comprising separating a stream comprising O₂ and N₂ to form the oxygen-containing stream and a nitrogen-containing stream, the oxygen-containing stream comprising 55 vol % or more of O₂ prior to combining the oxygen-containing stream with at least one of the hydrocarbon-containing stream and the steam.

4. The method of claim 2, further comprising exposing at least a portion of the nitrogen stream and at least a portion of the gas phase product to a catalyst under ammonia synthesis conditions to form ammonia.

5. The method of claim 4, further comprising exposing at least a first portion of the ammonia to a urea synthesis catalyst in the presence of CO₂ under urea synthesis conditions to form urea.

6. The method of claim 5, further comprising exposing at least a portion of the urea to a catalyst in the presence of sulfur to form a fertilizer product.

7. The method of claim 6, further comprising separating H₂S from the gas phase product to form a desulfurized gas phase product and a sulfur-containing product, and wherein at least a second portion of the ammonia is exposed to a catalyst in the presence of at least a portion of the sulfur-containing product to form the fertilizer product.

8. The method of claim 7, further comprising separating CO₂ from at least one of the gas phase product and the desulfurized gas phase product to form a synthesis gas stream and a CO₂-containing product.

9. The method of claim 8, wherein the at least a first portion of the ammonia is exposed to the urea synthesis catalyst in the presence of at least a first portion of the CO₂-containing product to form urea.

10. The method of claim 8, wherein a second portion of the CO₂-containing product is recycled to the gasifier as an additional diluent.

11. The method of claim 8, wherein the at least a portion of the gas phase product comprises at least a portion of the synthesis gas stream.

12. The method of claim 1, wherein passing at least a portion of the solid particles comprising deposited coke from the reactor to the gasifier comprises passing the at least a portion of the solid particles comprising deposited coke to a heater, and passing the at least a portion of the solid particles comprising deposited coke from the heater to the gasifier.

13. The method of claim 1, wherein passing at least a second portion of the partially gasified coke particles from the gasifier to the reactor comprises passing the at least a second portion of partially gasified coke particles to a heater, and passing the at least a second portion of the partially gasified coke particles from the heater to the reactor.

14. The method of claim 1, wherein the solid particles comprise coke.

15. A method for producing synthesis gas or products derived from synthesis gas, comprising:
exposing a feedstock comprising a T10 distillation point of 343° C. or more to a fluidized bed comprising solid particles in a reactor under thermal cracking conditions to form a 343° C.− liquid product, the thermal cracking conditions comprising 10 wt % or more conversion of the feedstock relative to 343° C., the thermal cracking conditions being effective for depositing coke on the solid particles;
introducing steam and a stream comprising O₂ and N₂ into a gasifier,
passing at least a portion of the solid particles comprising deposited coke from the reactor to the gasifier;
exposing the at least a portion of the solid particles comprising deposited coke to gasification conditions in the gasifier to form a gas phase product comprising H₂, N₂, CO, and CO₂ and partially gasified coke particles;
removing at least a first portion of the partially gasified coke particles from the gasifier;
passing at least a second portion of the partially gasified coke particles from the gasifier to the reactor;
separating, during a first time period, CO₂ from at least a portion of the gas phase product to form a dilute synthesis gas stream;
separating, during the first time period, N₂ from the dilute synthesis gas stream to form a nitrogen-containing stream and a synthesis gas stream;
exposing, during the first time period, at least a portion of the synthesis gas stream to a catalyst in a synthesis reactor to form a chemical product;
combining, during the first time period, a hydrocarbon-containing stream with the nitrogen-containing stream to form a low-BTU gas, at least a portion of the low-BTU gas being passed as a fuel or reagent to an additional process;
stopping, during a second time period, the operation of the synthesis reactor, and passing, during the second time period, at least a portion of the gas phase product to the additional process.

16. The method of claim 15, wherein the hydrocarbon-containing stream further comprises H₂.

17. The method of claim 15, further comprising separating, during the first time period, H₂S from the gas phase product to form a desulfurized gas phase product, the CO₂ being separated during the first time period from the desulfurized gas phase product.

18. The method of claim 15, further comprising separating, during the second time period, H₂S from the gas phase product prior to passing the at least a portion of the gas phase product to the additional process.

19. The method of claim 15, further comprising introducing a second hydrocarbon-containing stream into the gasifier, the gasification conditions further comprising conditions for at least one of gasifying and reforming hydrocarbons in the second hydrocarbon-containing stream in the presence of the steam.

20. The method of claim 15, wherein the chemical product comprises at least one of methanol and ammonia.
